(19) United States
(12) Patent Application Publication
(10) Pub. No.: US 2023/0000944 A1
(43) Pub. Date: Jan. 5, 2023

(54) Skin Protectant Paste

(71) Applicant: Kenneth Okafor, Eagan, MN (US)

(72) Inventor: Kenneth Okafor, Eagan, MN (US)

(21) Appl. No.: 17/364,804

(22) Filed: Jun. 30, 2021

(51) Int. Cl. A61K 38/12; A61K 31/4164; A61K 31/732; A61K 31/717; A61K 47/06; A61K 38/01

(52) U.S. Cl. CPC A61K 38/12; A61K 31/4164; A61K 31/732; A61K 31/717; A61K 47/06; A61K 38/014

(57) ABSTRACT

An article of manufacture for providing a skin protective paste is disclosed.

### TECHNICAL FIELD

This application relates in general to an article of manufacture for providing healthy skin treatment, and more specifically, to a skin protective paste providing both a topical antifungal and an antibiotic.

### BACKGROUND

Most available over the counter (OTC) pastes for diaper rash do not contain a topical antifungal and an antibiotic. The medical community/doctors/American Academy of Pediatrics actively recommends against using extended antibiotics in OTC topical treatments for diaper rash to prevent the development of resistance to antibiotics. Pastes typically made according to an individual prescriber's orders are even harder for patients to obtain. These prescribed pastes have never included a topical antibiotic for additional protection.

Therefore, a need exists for an article of manufacture for providing a skin protective paste providing both a topical antifungal and an antibiotic. The present invention attempts to address the limitations and deficiencies in prior solutions according to the principles and example embodiments disclosed herein.

### SUMMARY

In accordance with the present invention, the above and other problems are solved by providing a skin protective paste providing both a topical antifungal and an antibiotic according to the principles and example embodiments disclosed herein.

In one embodiment, the present invention is a skin protective paste providing both a topical antifungal and an antibiotic. The paste comprises stoma powder 35 gm, clotrimazole 1.2 gm, bacitracin zinc 500 u, polymyxin B sulfate 10,000 u, and modified white petrolatum sufficient to make 120 gm of the finished product (topical paste).

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description.

### DETAILED DESCRIPTION

This application relates in general a system and method for providing protection from diaper rash and bed sores, and more specifically, to an article of manufacture for providing a skin protective paste providing both a topical antifungal and antibiotics according to the present invention.

Various embodiments of the present invention will be described herein. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two components recited in succession may in fact be executed substantially concurrently or may sometimes be utilized in the reverse order, depending upon the functionality and acts involved.

The terms “individual” and “user” refer to an entity, e.g., a human, using an article of manufacture providing a skin protective paste with both a topical antifungal and an antibiotic associated with the invention. The term user herein refers to one or more users.

The term “invention” or “present invention” refers to the invention being applied for via the patent application with the title “A Skin Protectant Paste.” Invention may be used interchangeably with paste.

In general, the present disclosure relates to an article of manufacture for providing a skin protective paste providing both a topical antifungal and antibiotic according to the present invention. A paste may be made in various pharmacies that are primarily for treatment of a diaper rash. A formulation of the present invention improves these pastes to provide two additional topical antibiotics and a different antifungal agent. All available OTC for topical use.

The paste according to the present invention is made of:
1. Stoma powder 35 gm;
2. Clotrimazole 1.2 gm;
3 Bacitracin zinc 500 u;
4. Polymyxin B sulfate 10,000 u; and
5. Modified white petrolatum (QS) sufficient to make 120 gm. of the finished product (paste).

A 4 oz (120 grams) of the product will contain each amount of the ingredient as listed above along with enough of Modified white petrolatum sufficient to produce 120 grams of the final product, i.e., QS. As stated before, the paste is formed by combining stoma powder with the modified white petrolatum (may contain about 41% of pure white petrolatum), then the other ingredients are added in the specified amount, then more Modified petrolatum is added up to 120 grams of the final product.

An example of a modified 41% of pure white petrolatum is a product available in the market as Aquaphor. A paste could be made by combining stoma powder and 100% white petrolatum (Vaseline), but the paste is too greasy and lacks “cosmetic elegance”. Modifying pure white petrolatum may be created using several emulsifiers. The main purpose of using a base of modified pure white petrolatum to form the paste when combined with stoma is to lessen the greasiness and make the product more elegant.

The present invention will be the first readily available (OTC) paste that really offers a broad-spectrum protection not only for diaper rash but also for bedsores and diabetic foot ulcers. The pastes may be made in a pharmacy. Existing skin treating pastes are dispensed in traditional ointment jars and are scooped with a user's fingers to apply to an affected area. The present invention may be dispensed in specialized jar or traditional ointment jars or tubes.

Similar products intended for diaper rash are available and typically referred to as butt paste, poop coop etc. The OTC products butt pastes are basically zinc oxide ointment, while those made in extemporaneous compounding have closer resemblance to the invention. Because of the intended use for mainly diaper rash, this product is typically not made as a solution, suspension, gel. However, some similar products made in extemporaneous compounding use a formula that contains mineral oil, baby powder, stoma powder and a thick cream like Eucerin™. In this case, the final product may be classified as a skin protective cream, because the largest ingredient, Eucerin™, is a cream. The predominant formula in extemporaneous compounding sequel to a prescription order is a stoma powder or paste, Aquaphor™, and nystatin ointment. In this case, the final product will be classified as an ointment paste because the largest ingredient, Aquaphor™, is an ointment.

Even though particular combinations of features are recited in the present application, these combinations are not intended to limit the disclosure of the invention. In fact, many of these features may be combined in ways not specifically recited in this application. In other words, any of the features mentioned in this application may be included in this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise.

What is claimed is:

1. A solution for providing a skin protective paste, the solution comprises:
Stomahesive powder 35 gm;
Clotrimazole 1.2 gm;
Bacitracin zinc 500 u;
Polymyxin B sulfate 10,000 u; and
Modified white petrolatum qs 120 gm.

2. The solution according to claim 1, wherein the modified white petrolatum is about 41% pure white petrolatum added in sufficient amount to make a 4 oz (120 grams) of finished skin protectant paste
